(19) United States
(12) Patent Application Publication
(10) Pub. No.: US 2012/0301506 A1
(43) Pub. Date: Nov. 29, 2012

(54) Oncolytic Virus as an Inducer for Innate Antitumor Immunity

(76) Inventors: XIAOLIU ZHANG, HOUSTON, TX (US); XINPING FU, HOUSTON, TX (US)

(21) Appl. No.: 13/299,905

(22) Filed: Nov. 18, 2011

Related U.S. Application Data
(60) Provisional application No. 61/416,705, filed on Nov. 23, 2010.

(51) Int. Cl. A61K 35/76; A61P 35/00

(52) U.S. Cl. 424/281.1

(57) ABSTRACT

The present invention is directed to the administration of FusOn-H2, an HSV derived oncolytic virus, to treat tumor cells that are resistant to the lytic effect of the virus. Administration of FusOn-H2 induces the patient's innate immune responses to tumor cells via neutrophils, which are able to destroy tumors efficiently when they migrate to the tumor mass. With the induced innate antitumor immunity, FusOn-H2 is effective at eradicating tumors even when it is used at very low doses.

### CROSS-REFERENCES TO RELATED APPLICATIONS

This application claims priority to provisional application No. 61/416,705 filed on Nov. 23, 2010, which is herein incorporated by reference in its entirety.

### GOVERNMENTAL SPONSORSHIP

The U.S. Government has a paid-up license in this invention and the rights in limited circumstances to require the patent owners to license others on reasonable terms as provided for by the terms of grant Nos. 7R01CA132792-03 and 7R01CA106671-07 awarded by the National Institute of Health. The Government has certain rights in this invention.

### FIELD OF THE INVENTION

The present invention relates to a novel method to inducing the host's innate immune responses to tumor cells, and such method represents a new strategy for the treatment of malignant tumors. More specifically, the increased immune response is achieved when FusOn-H2 virotherapy is used in tumors established from tumor cells that are resistant to the lytic effect of this virus. The major components of the induced innate antitumor immunity are neutrophils, which are able to destroy tumors efficiently when they migrate to the tumor mass. With the induced innate antitumor immunity, FusOn-H2 is effective at eradicating tumors even when it is used at very low doses.

### BACKGROUND OF THE INVENTION

A persistent observation of many emerging cancer treatments is that their beneficial effects extend only to a subset of patients. This phenomenon tends to be more common with biotherapeutic interventions such as immunotherapy and gene therapy. For example, studies by Morgan et al. showed that only 2 of 15 patients receiving infusions of their own modified T-cells responded with clearly objective regressions of metastatic melanoma (Morgan et al., Cancer regression in patients after transfer of genetically engineered lymphocytes, Sci. 314, 126-9 (2006).). With a few notable exceptions, such as the strong link between a mutated epidermal growth factor (EGFR) gene and clinical responses to its tyrosine kinase inhibitor Iressa (Lynch, et al. Activating mutations in the epidermal growth factor receptor underlying responsiveness of non-small-cell lung cancer to gefitinib, N. Engl. J. Med. 350, 2129-39 (2004); Paez et al., EGFR mutations in lung cancer: correlation with clinical response to gefitinib therapy, Sci. 304, 1497-500 (2004)), the mechanisms accounting for these unique responders to cancer biotherapy remain poorly understood. New insight into these mechanisms could greatly accelerate progress in the development of effective biotherapeutic agents for use in cancer patients.

Virotherapy is a strategy in which a virus that preferentially replicates in tumor cells is applied either locally or systemically to lyse such cells (Parato et al., Recent progress in the battle between oncolytic viruses and tumours, Nat. Rev. Cancer 5, 965-76 (2005)). Unlike typical forms of gene-based cancer therapy, oncolytic viruses are thought to kill tumor cells directly through selective replication/cytolysis and consequent spread to surrounding tumor tissues. These properties represent a major advantage over the inherent inefficiency of gene delivery and the resultant limited tumor cell killing effect of conventional gene therapies. Several viruses, including adenovirus (Bischoff et al., An adenovirus mutant that replicates selectively in p53-deficient human tumor cells, Sci. 274, 373-6 (1996)), herpes simplex virus (Martuza et al., Experimental therapy of human glioma by means of a genetically engineered virus mutant, Sci. 252, 854-6 (1991)), retrovirus (Logg et al., A uniquely stable replication-competent retrovirus vector achieves efficient gene delivery in vitro and in solid tumors, Hum. Gene. Ther. 12, 921-32 (2001)), vaccinia virus (McCart, Systemic cancer therapy with a tumor-selective vaccinia virus mutant lacking thymidine kinase and vaccinia growth factor genes, Cancer. Res. 61, 8751-7 (2001)), measles virus (Peng et al., Intraperitoneal therapy of ovarian cancer using an engineered measles virus, Cancer Res. 62, 4656-62 (2002)) and vesicular stomatitis virus (Stojdl et al., Exploiting tumor-specific defects in the interferon pathway with a previously unknown oncolytic virus, Nat. Med. 6, 821-5 (2000)) have been modified for oncolytic purposes. These viruses can be derived either from naturally occurring viruses that preferentially target tumor cells (Stojdl et al., Exploiting tumor-specific defects in the interferon pathway with a previously unknown oncolytic virus, Nat. Med. 6, 821-5 (2000)) or from genetically engineered viruses that target cancer cells by a defined molecular mechanism (Glasgow et al., Transductional and transcriptional targeting of adenovirus for clinical applications, Curr. Gene Ther. 4, 1-14 (2004); Martuza et al., Experimental therapy of human glioma by means of a genetically engineered virus mutant, Sci. 252, 854-6 (1991); McCormick et al., Cancer-specific viruses and the development of ONYX-015, Cancer Biol. Ther. 2, S157-60 (2003); Van der Poel et al., Epidermal growth factor receptor targeting of replication competent adenovirus enhances cytotoxicity in bladder cancer, J. Urol. 168, 266-72 (2002)). Despite only a relatively short history of research and development, several oncolytic viruses are being tested in clinical trials against tumors of different tissue origins; in general, they have shown excellent safety profiles and some have produced indications of efficacy (Bell, Oncolytic viruses: what's next? Curr. Cancer Drug Targets 7, 127-31 (2007)). However, as with many other biotherapeutic approaches, the clinical utility of virotherapy is restricted by the generally small group of patients with favorable responses. In one recent clinical trial, 26 patients were treated with an oncolytic virus derived from a type I herpes simplex virus (HSV-1), but only three had favorable responses (Hu et al., A phase I study of OncoVEXGM-CSF, a second-generation oncolytic herpes simplex virus expressing granulocyte macrophage colony-stimulating factor, Clin. Cancer Res. 12, 6737-47 (2006)). This and similar outcomes underscore the need to understand why some tumors (but not others) respond well to treatment with oncolytic viruses. The present invention offers a solution to widen the efficacy of virotherapy to a broader group of patients.

We recently developed an oncolytic virus based on a type II herpes simplex virus (HSV-2) by deleting the N-terminal region of the ICP10 gene from the viral genome (Fu et al., A Mutant Type 2 Herpes Simplex Virus Deleted for the Protein Kinase Domain of the ICP10 Gene Is a Potent Oncolytic Virus, Mol. Ther. 13, 882-90 (2006). Designated FusOn-H2, it has multiple antitumor mechanisms and has shown potent oncolytic activity against tumor cells of different tissue origins (Fu et al., A Mutant Type 2 Herpes Simplex Virus Deleted for the Protein Kinase Domain of the ICP10 Gene Is a Potent Oncolytic Virus, Mol. Ther. 13, 882-90 (2006); Fu et al., Effective treatment of pancreatic cancer xenografts with a conditionally replicating virus derived from type 2 herpes simplex virus, Clin. Cancer Res. 12, 3152-57 (2006); Fu et al., An oncolytic virus derived from type 2 herpes simplex virus has potent therapeutic effect against metastatic ovarian cancer, Cancer Gene Ther. 14, 480-7 (2007). Yet, several tumor cell lines that we have screened in vitro show almost resistant to the replication of FusOn-H2 and thus its oncolytic effect. This suggests that patients whose tumor cells are nonpermissive to FusOn-H2 replication in vitro would be unresponsive to FusOn-H2 virotherapy. The present invention shows that an intrinsically strong interferon response activity underlies the resistance of murine and human tumors to FusOn-H2 lytic activity, but does not preclude a favorable therapeutic response. To the contrary, treatment of implanted tumors with FusOn-H2 virotherapy led to their infiltration and destruction by neutrophils, indicating that FusOn-H2 can function as an inducer for a host's innate antitumor immunity. The current invention clearly demonstrates that using an oncolytic virus to induce a host's innate antitumor immunity provides a new strategy for the treatment of malignant tumors.

### SUMMARY OF THE INVENTION

The present invention is a novel method of treating cancer in an oncolytic virotherapy by administering FusOn-H2 to tumor cells that are resistant to the lytic effect of the virus. Oncolytic virotherapy has shown substantial promises as an alternative therapeutic modality for solid tumors in both preclinical studies and clinical trials. The main therapeutic activity of virotherapy derives from the direct lytic effect associated with virus replication and the induction of host immune responses to the infected tumor cells. As a result, prior to the present invention, studies suggested that patients having nonpermissive tumor cells would likely be unresponsive to FusOn-H2 virotherapy. However, the present invention demonstrates that oncolytic viruses can function as a potent inducer for a host's innate antitumor immunity, including in cells that are resistant to the lytic effect of the oncolytic viruses. Thus, using oncolytic virus to induce host's innate antitumor immunity provides a new strategy for the treatment of malignant tumors.

### BRIEF DESCRIPTION OF THE DRAWINGS

FIG. 1. Wild variation of FusOn-H2 replication in tumor cells of different tissue origins. Cells were seeded in 24-well plates in duplicate and infected with FusOn-H2 at 0.1 pfu per cell for 1 h. Cells were washed and harvested with or without 24 h incubation. The fold increase in viral replication was calculated by dividing the virus titer at 24 h after infection by the values of titer for the same cells harvested immediately after washing without incubation. The data are reported as means of triplicate experiments.

FIG. 2. Administration of FusOn-H2 effectively shrank established EC9706 tumors despite the inefficiency of its replication in this tumor cell Tumors were initially established by implanting 5×10⁶ EC9706 cells in the right flank of nude mice. Once tumors reached the approximate size of 5 mm in diameter, they were injected with FusOn-H2 at a dose of 3×10⁶ or 6×10⁴ pfu. Tumors were measured weekly post-treatment, and the tumor growth rate was determined by dividing the tumor volume before treatment by the tumor volume after treatment.

FIG. 3. FusOn-H2 induces massive infiltration of neutrophils in the resistant tumors. Resistant EC9706 (a, b, d and e) or permissive 4T1 (c) tumor cells were implanted on the right flank (a, b, c, d) or on both flanks (e) of female nu/nu mice. Once tumors reached the approximate size of 5 mm in diameter, FusOn-H2 or PBS was injected into the tumors on the right flank, as indicated. Tumors were explanted on days 1, 2, 3 and 5 and sectioned for H&E staining The sections shown here represent day 2 after virus or PBS administration. Blue arrows indicate infiltration; the white arrow marks degenerating tumor cells.

FIG. 4. Qualitative and quantitative characterization of the infiltrating neutrophils. EC9706 tumors were established on the right flank of nude mice and injected with 3×10⁶ pfu of FusOn-H2 (a, d) or the same FusOn-H2 that had been activated by UV radiation (b, e), or PBS (c). Tumors were explanted two days later and divided into halves; one half for preparation of frozen sections for examining GFP expression under a fluorescent microscope (d, e) and the other half for immunohistochemical staining of neutrophils (a-c). The infiltrating neutrophils from a-c were quantitated by counting 10 microscopic fields (40×) and the average numbers are plotted in f. *p<0.01 vs. inactivated FusOn-H2.

FIG. 5. Neutrophils isolated from the treated tumor cells can efficiently kill tumor cells when assayed in vitro. Neutrophils were isolated from either established EC9706 tumors that had been treated with FusOn-H2 (TN) or from peritoneal cavity that had been injected with EC9706 cells infected with FusOn-H2 (VPN) or mock infected (CPN). The purified neutrophils were then mixed with EC9706 cells at the indicated ratios and cytolysis was determined 24 h later.

FIG. 6. The neutrophil infiltration is linked to the endogenous interferon response activity in these tumor cells. Tumor cells were transfected with 1 μg of pJ-ISRE-SEAP by lipofectamine and the supernatants were collected either at 24 h later (a) or periodically (b) at the indicated times for quantification of SEAP. The results in b were obtained from EC9706 cells.

FIG. 7. Induction of neutrophil infiltration by FusOn-H2 is a generalized phenomenon that can be detected in other resistant tumors. 2×10⁵ B16 cells were implanted subcutaneously to the right flank of C57BL/6 mice. Once tumors reached the approximate size of 5 mm in diameter, they were injected with 3×10⁶ pfu of either FusOn-H2 or Baco-1 (an HSV-1-based oncolytic virus) or PBS. Tumors were explanted two days later and tumor sections were prepared for H&E staining The infiltrating neutrophils were quantitated by counting 10 fields (40×) under a microscope and the average numbers are plotted. *p<0.01 vs. Baco-1.

### DETAILED DESCRIPTION OF THE INVENTION

The present invention relates to a novel method to significantly increase the host's innate immune responses to infected tumor cells, and such method represents a new strategy for the treatment of malignant tumors. More specifically, the increased immune response is achieved when FusOn-H2 virotherapy is used in tumors established from tumor cells that are resistant to the lytic effect of this virus. The major components of the induced innate antitumor immunity are neutrophils, which are able to destroy tumors efficiently when they migrate to the tumor mass. This allows FusOn-H2 to show satisfactory antitumor effect even when it is used at a very low dose.

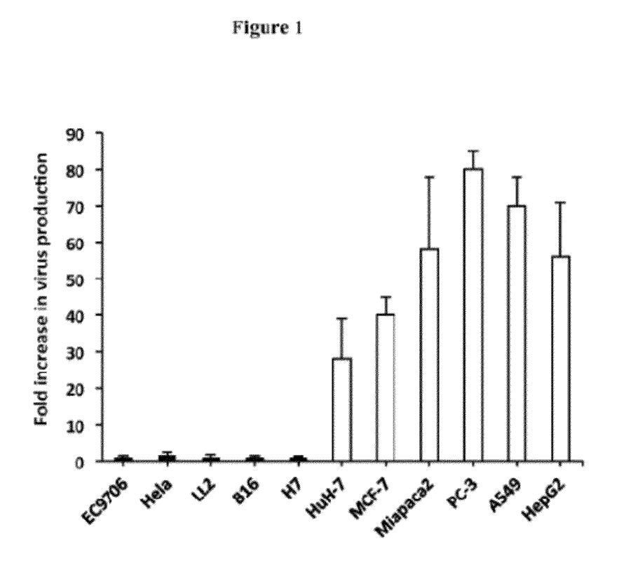

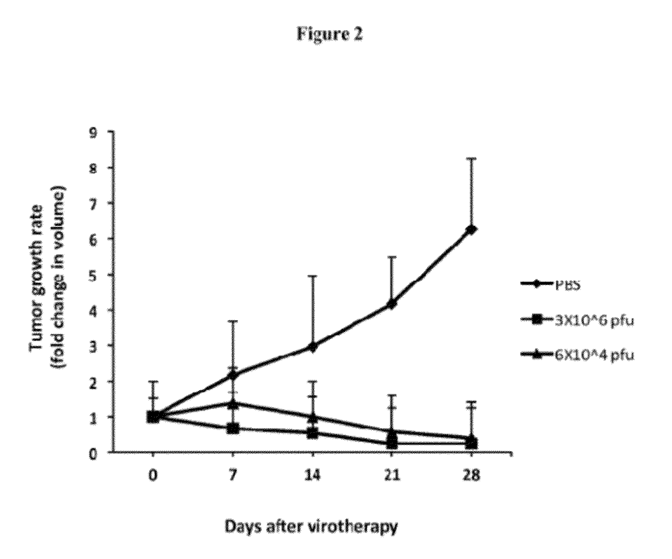

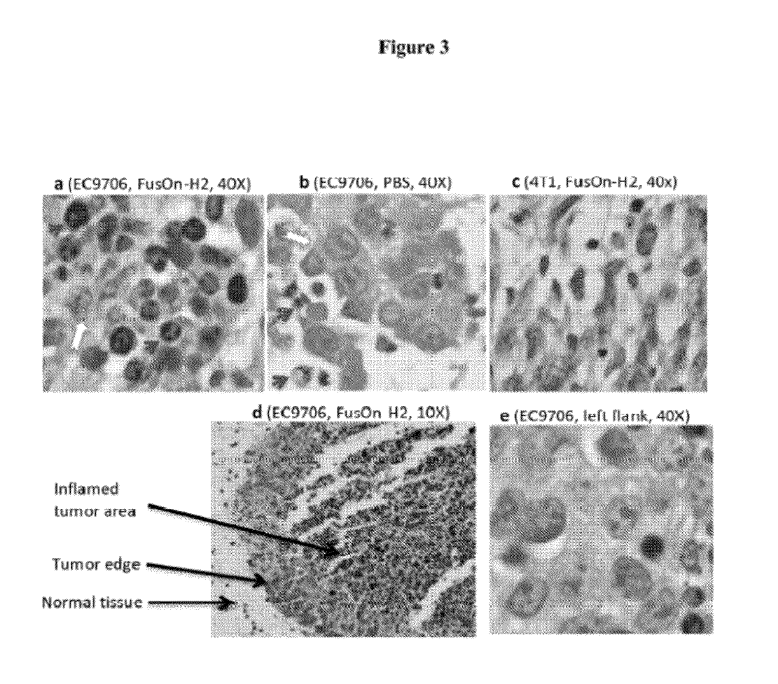

In a preferred embodiment of the invention, FusOn-H2 replicates differentially in tumor cells of different tissue origins (FIG. 1). Since the construction of FusOn-H2, it has been characterized in more than two dozen tumor cell lines derived from different tissues of both humans and mice. FusOn-H2 efficiently lysed most of the tumor cells that were screened and effectively shrank tumors established from these cells when injected either locally or systemically (Fu et al., A Mutant Type 2 Herpes Simplex Virus Deleted for the Protein Kinase Domain of the ICP10 Gene Is a Potent Oncolytic Virus, Mol. Ther. 13, 882-90 (2006); Fu et al., Effective treatment of pancreatic cancer xenografts with a conditionally replicating virus derived from type 2 herpes simplex virus, Clin. Cancer Res. 12, 3152-57 (2006); Fu et al., An oncolytic virus derived from type 2 herpes simplex virus has potent therapeutic effect against metastatic ovarian cancer, Cancer Gene Ther. 14, 480-7 (2007)). However, results also showed that approximately 20% of the tumor cell lines were resistant to FusOn-H2 replication. In contrast to the fully permissive tumor cells (see HuH-7, MCF-7, Miapaca2, PC-3, A549, and Hep G2 in FIG. 1) in which the input virus replicated as much as 100-fold within 48 h after infection, the yield of FusOn-H2 replication in each of five tumor cell lines representing esophageal carcinoma (EC9706), cervical cancer (Hela), lung carcinoma (LL2), pancreatic cancer (H7) and melanoma (B16), barely increased over the same time period (FIG. 1). Prior art shows that, in most cases, the oncolytic virus can infect tumor cells, as indicated by the expression of green fluorescent protein (GFP) gene after it was inserted into the viral genome during its construction (Fu et al., A Mutant Type 2 Herpes Simplex Virus Deleted for the Protein Kinase Domain of the ICP10 Gene Is a Potent Oncolytic Virus, Mol. Ther. 13, 882-90 (2006)). The blockage of virus growth in these tumor cells (e.g., EC9706, Hela, LL2 and B16) is known to occur mainly during virus replication. Because the therapeutic effect of an oncolytic virus is believed to depend mainly on its ability to replicate and spread, the results published to date indicate that FusOn-H2 would be largely ineffective against tumors established from these cell lines. However, the results presented herein, which is one of the objects of the present invention, reveal quite the opposite; that is, FusOn-H2 still has the ability to shrink tumors established from resistant tumor cells by inducing the infiltration of neutrophils in the tumor's stroma (as shown in FIGS. 2 and 3).

In another embodiment of the present invention, FusOn-H2 has a high therapeutic effect against implanted tumors established from cancer cell lines that are known to be resistant to viral replication. As tumor cell resistance to viral replication generally predicts a poor response to virotherapy, tumors established from such resistant cells were initially excluded from in vivo evaluation of the antitumor effects of FusOn-H2. Recently, however, several resistant tumor cell lines were included in in vivo experiments, primarily as negative controls. Surprisingly, a single injection of FusOn-H2 at 3×10⁶ plaque-forming units (pfu) produced a dramatic antitumor effect, nearly eradicating tumors established from implants of EC9706 cells, which are resistant to FusOn-H2 replication (FIG. 2). This effect was essentially duplicated when the virus dose was reduced 50-fold, to as low as 6×10⁴ pfu (FIG. 2). Together, these observations show that FusOn-H2 destroys resistant tumor cells in vivo through mechanisms other than direct oncolysis.

In another embodiment of the present invention, FusOn-H2 induces an infiltration of neutrophils into resistant tumor cells. To account for the unexpected antitumor effects of FusOn-H2 virotherapy, tumors from EC9706 or 4T1 cells (a murine mammary tumor line that is significantly more permissive than EC9706 to FusOn-H2 replication) were initially established. After their injection with FusOn-H2, the tumors were harvested at days 1, 2, 3 and 5 for histological examination. The results revealed a infiltration of neutrophils in EC9706 tumors treated with FusOn-H2 as indicated by the blue arrows on FIG. 3. The inner areas of the tumors were almost entirely filled with neutrophils; the few remaining tumor cells did not appear healthy, as indicated by the white arrow in FIG. 3a. Tumor cells near the periphery seemed viable and formed a ring surrounding the inflamed interior (FIG. 3d). Infiltrating neutrophils were much less common in EC9706 tumors treated with PBS (FIG. 3b) and were virtually undetectable in 4T1 tumors, which showed obvious oncolytic effects due to robust FusOn-H2 replication (FIG. 3c). In a subsequent experiment, EC9706 tumor cells were inoculated into both flanks and tumors on the right flank with FusOn-H2 were treated. The treated tumors shrank, but tumor growth on the opposite flank was not affected. Histological examination of the untreated tumors did not reveal any increases in neutrophil infiltration (FIG. 3e), indicating that the infiltration of neutrophils was a regional effect that was directly associated with virus infection.

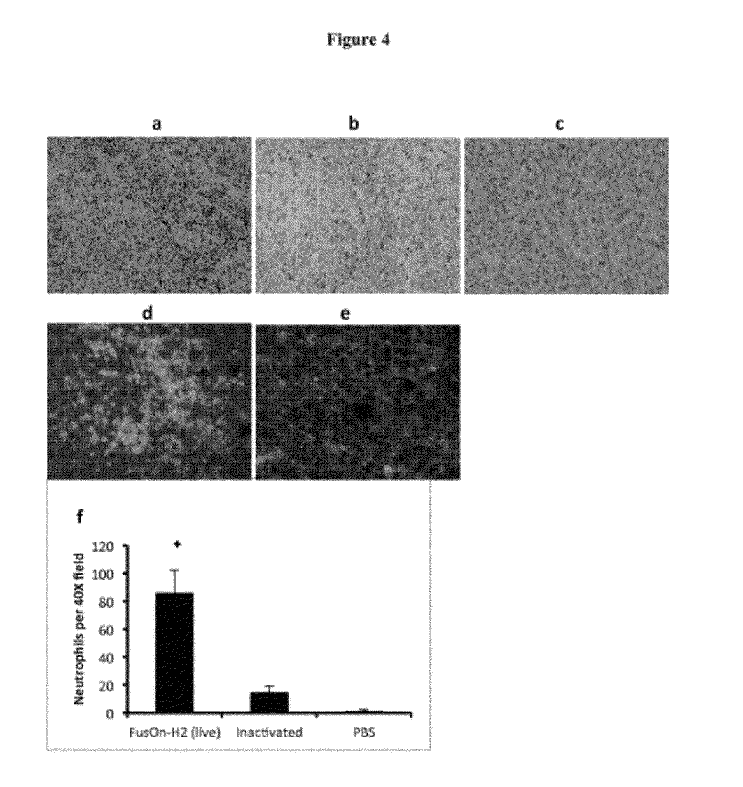

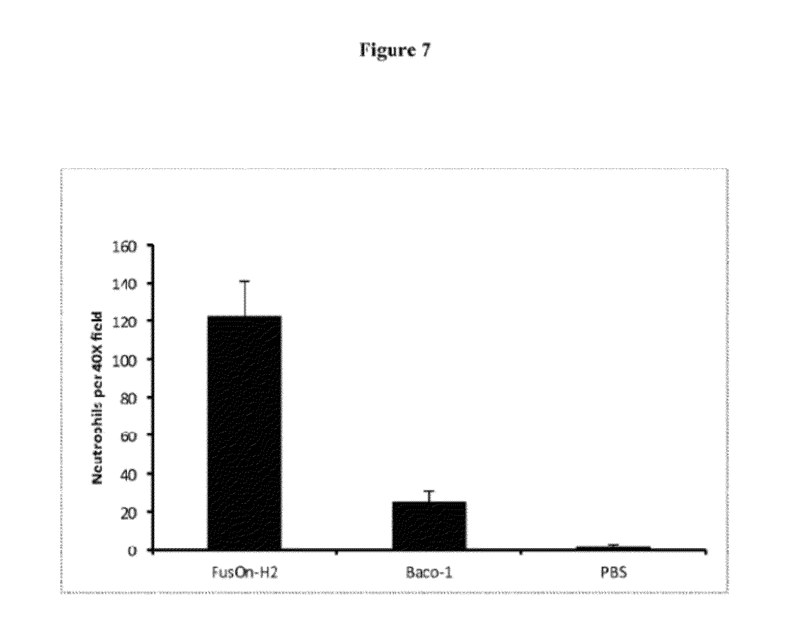

In another embodiment of the invention, infiltrating neutrophils are characterized. Established EC9706 tumors were injected with either 3×10⁶ pfu of FusOn-H2 or the same amount of virus that had been inactivated by UV radiation, or PBS. Tumors were explanted 2 days later and divided into halves. One half was used for preparation of frozen sections for visualization of virus infection by examining GFP expression under a fluorescent microscope. As FusOn-H2 contains the GFP gene, the virus infectivity could be conveniently determined by this method. The other half of tumors was used for preparation of paraffin sections for immunohistochemical staining of neutrophils. The results were shown in FIG. 4. The micrographs, taken at a low magnitude (10×) from sections immunohistochemically stained for neutrophils, showed that there was a widespread neutrophil infiltration in tumors treated with FusOn-H2 (FIG. 4a). However, the extent of neutrophil infiltration was drastically reduced in tumors treated with the inactivated virus (FIGS. 4b and 4f), indicating that virus infectivity was probably necessary for the induction of neutrophil infiltration. Neutrophils were not readily visible in untreated tumors, suggesting that these tumors were not intrinsically associated with neutrophil infiltration. Similar neutrophil infiltration was also detected in tumors established from another resistant tumor cells, B16 murine melanoma, after FusOn-H2-virotherapy as shown in FIG. 7.

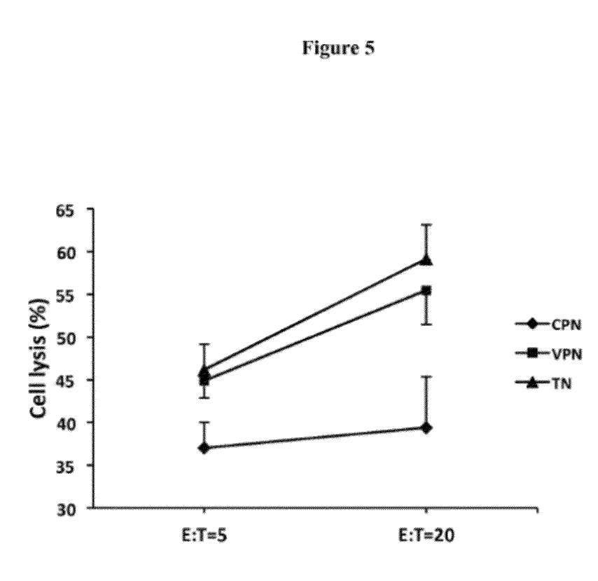

In another embodiment of the present invention, neutrophils isolated from tumor tissues or from peritoneal cavity demonstrate killing activity. To examine the effect of the infiltrating neutrophils on EC9706 cells more directly, an in vivo experiment is performed similar to that illustrated in FIG. 3. After harvesting infiltrating neutrophils from established tumors at 2 days post-treatment, they are immediately mixed with EC9706 tumor cells at different ratios and measured cytolysis 24 h later (FusOn-H2 was undetectable in the purified neutrophils). The neutrophils retrieved from the FusOn-H2-treated EC9706 tumors had a significantly higher killing activity against EC9706 tumor cells than did those isolated from untreated tumors, as illustrated in FIG. 5. These results demonstrate a critical difference in cytolytic capacity between neutrophils in FusOn-H2-treated versus untreated EC9706 tumors.

An innate immune response dominated by neutrophils, as demonstrated herein, offer some distinct and unique advantages over what is commonly known as adaptive immunity. First, the infiltration of tumors by neutrophils after treatment with FusOn-H2 is uniform, as demonstrated in FIG. 3. By contrast, during adaptive immune responses, T effector cells are usually found at low frequencies in tumor tissues, which may have limited their antitumor efficacy. Indeed, for adaptive antitumor immunity to be successful, substantial expansion of the initially generated tumor-specific T cells is crucial (Van Heijst et al., Recruitment of antigen-specific CD8+ T cells in response to infection is markedly efficient, Sci. 325, 1265-9 (2009). In most cases, however, T effector cell proliferation has proved extremely inefficient within the tumor microenvironment, probably accounting, at least in part, for the disappointing overall results from an array of clinical trials of T cell-based immunotherapy. Another major advantage of the use of neutrophils over T cells in tumor destruction is that the former has the ability to liquefy the entire tumor tissues, which include tumor cells and tumor stromas such as collagen fibrils, stromal cells, lymphatics and capillaries (Jain, Transport of molecules, particles, and cells in solid tumors, Annu Rev. Biomed Eng. 1, 241-63 (1999)). By contrast, T cells can only lyse tumor cells and their effects are frequently limited or actively inhibited by the remaining tumor stroma. Thus, given the relative ease with which large numbers of tumor-killing neutrophils were recruited to tumor sties in this study, we suggest that FusOn-H2 virotherapy represents a unique strategy for enhancing the impact of immunotherapy against certain subgroups of tumors.

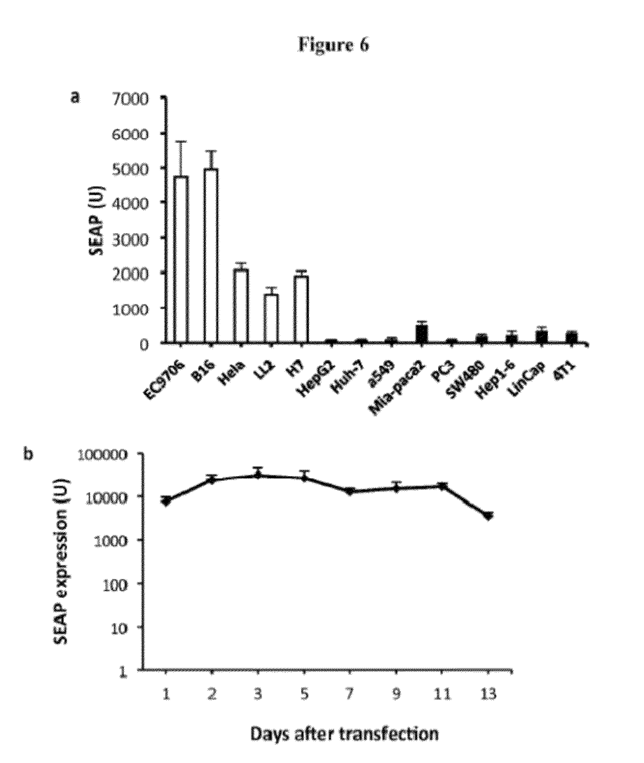

In another embodiment of the invention, there is an endogenous interferon response activity in tumor cells with resistance to FusOn-H2 replication. The observation that only FusOn-H2-resistant tumors showed neutrophil infiltration after virotherapy indicates an intrinsic biological difference between the resistant and nonresistant tumors. To pursue this notion, the interferon response status of the tumor cells is evaluated, as this response functions as a critical innate antiviral mechanism and could explain the failure of FusOn-H2 to replicate well in some tumor lines but not others. For this purpose, a test plasmid, pJ-ISRE-SEAP, is constructed in which the gene encoding the secreted form of alkaline phosphatase (SEAP) is driven by a minimal promoter linked to 3 tandem repeats of the interferon-stimulated response element (ISRE), derived from the ISG56 promoter region. When transfected into tumor cells, this construct enabled measurement of SEAP levels in the culture medium and to monitor the cells' interferon response activity. FIG. 6a shows the results of transfecting pJ-ISRE-SEAP into the five lines of tumor cells that showed resistance to FusOn-H2 as well as a panel of tumor cells that are permissive to the virus. Supernatants were collected and the secreted SEAP quantified at different time points after transfection. All five resistant cell lines had much higher levels of SEAP secretion than did the permissive lines (FIG. 6a). Among the five resistant lines, EC9706 (human esophageal carcinoma) and B16 (murine melanoma) showed an extremely high level of ISRE activity. SEAP secretion by EC9706 cells is also monitored for an extended time, demonstrating that it peaked on day 5 after transfection. Thereafter, it declined slightly but remained at a relatively high level for up to 2 weeks, the longest time span monitored (FIG. 6b).

While the invention described here specifically focuses on a novel method to significantly increase the host's immune responses by the introduction of neutrophils into tumor cells infected with FusOn-H2, one of ordinary skills in the art, with the benefit of this disclosure, would recognize the extension of the approach to other combinatorial treatment regimens for the treatment of resistant tumors in a clinical setting.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no embodiments herein are intended to limit the scope of the claim of this invention. It is therefore evident that the particular illustrative embodiments disclosed above may be altered or modified and all such variations are considered within the scope and spirit of the present invention.

### EXAMPLES

### Example 1

### Tumor Cells of Different Tissue Origins Show Wild Variation in their Sensitivity to the Replication of Oncolytic HSV FusOn-H2

FusOn-H2 was characterized in more than a dozen tumor cell lines derived from different tissues of both humans and mice. FusOn-H2 efficiently lysed many of the tumor cells that were screened. However, approximately 20% of the tumor cell lines were resistant to FusOn-H2 replication. In contrast to the fully permissive tumor cells, in which the input virus replicated as much as 100-fold within 48 h after infection, the yield of FusOn-H2 in each of five tumor cell lines representing esophageal carcinoma, cervical cancer, lung carcinoma, melanoma and pancreatic cancer, barely increased over the same time period. In most cases, the oncolytic virus can infect the tumor cells, as indicated by the expression of green fluorescent protein (GFP) gene, which was inserted into the viral genome during its construction. The blockage of virus growth in these tumor cells mainly occurred during virus replication. Because the therapeutic effect of an oncolytic virus is believed to depend mainly on its ability to replicate and spread, these findings indicate that FusOn-H2 would be largely ineffective against tumors established from these cell lines.

### Example 2

### FusOn-H2 is Effective Against Implanted Tumors Established from Some of the Cancer Cell Lines Resistant to Viral Replication

As tumor cell resistance to viral replication generally predicts a poor response to virotherapy, these tumor cells were usually excluded from in vivo efficacy evaluation of virotherapy. However, when we elected to include several resistant tumor cell lines in our in vivo experiments, the results turned out to be very surprising. A single injection of FusOn-H2 at 3×10⁶ plaque-forming units (pfu) produced a dramatic antitumor effect, nearly eradicating tumors established from implants of EC9706 cells, which are resistant to FusOn-H2 replication. This effect was essentially duplicated when the virus dose was reduced 50-fold, to as low as 6×104 pfu. Other than tumor disappearance, the animals showed no sign of toxicity during the virotherapy. These observations suggest that FusOn-H2 destroyed the highly resistant tumor cells in vivo through some unique mechanisms other than direct oncolysis.

### Example 3

### FusOn-H2 Induces Massive Infiltration of Neutrophils into Resistant Tumors

To account for the unexpected antitumor effects of FusOn-H2 virotherapy, we initially established tumors from EC9706 or 4T1 cells (a murine mammary tumor line that is significantly more permissive than EC9706 to FusOn-H2 replication but otherwise is similar to EC9706 in that they both form tumors aggressively once implanted into mice). After their injection with FusOn-H2, the tumors were harvested at days 1, 2, 3 and 5 for histological examination. The results revealed a massive infiltration of neutrophils in EC9706 tumors treated with FusOn-H2. The inner areas of the tumors were almost entirely filled with neutrophils; the few remaining tumor cells did not appear healthy. Tumor cells near the periphery seemed viable and formed a ring surrounding the inflamed interior. In contrast, infiltrating neutrophils were much less common in EC9706 tumors treated with PBS and were virtually undetectable in 4T1 tumors, which showed obvious oncolytic effects due to robust FusOn-H2 replication.

### Example 4

### Neutrophils Retrieved from FusOn-H2 Treated Tumors can Lyse Tumor Cells When Assayed In Vitro

To examine the effect of the infiltrating neutrophils on EC9706 cells more directly, we performed an additional in vivo experiment in which neutrophils were retrieved from EC9706 tumors treated with FusOn-H2 at 2 days post-treatment. The neutrophils were mixed with EC9706 tumor cells at different ratios and measured cytolysis 24 h later (FusOn-H2 was undetectable in the purified neutrophils). The neutrophils retrieved from the FusOn-H2-treated EC9706 tumors had a significantly higher killing activity against EC9706 tumor cells than did those isolated from untreated tumors, demonstrating a critical difference in cytolytic capacity between neutrophils in FusOn-H2-treated versus untreated EC9706 tumors.

### Example 5

### Neutrophils Retrieved from FusOn-H2 Treated Tumors can Actively Migrate Towards Tumor Cells when Assayed In Vitro

We also measured the effect of FusOn-H2-infected tumor cells on the migration ability of neutrophils in an in vitro experiment. Freshly isolated neutrophils and tumor cells of different preparations (mock-infected, infected with 1 pfu/cell of FusOn-H2 or UV-inactivated FusOn-H2) were seeded in matrigel invasion chambers for cell migration assay as described. The results show that significantly more neutrophils were migrating toward the well seeded with FusOn-H2-infected EC9706 cells than to the wells seeded with two permissive cell lines, 4T1 and MD-MBA-435. As compared with the mock-infected cells, cells infected with UV-inactivated FusOn-H2 can increase neutrophil migration. However, to achieve the maximal chemoattractant effect, full infectivity of the virus is required.

### Example 6

### Possible Link Between a Strong Endogenous Interferon Response Activity in Tumor Cells Resistance to FusOn-H2 Replication and the Induced Neutrophil Infiltration

The observation that only FusOn-H2-resistant tumors showed massive neutrophil infiltration after virotherapy indicates an intrinsic biological difference between the resistant and nonresistant tumors. To pursue this notion, we first evaluated the interferon response status of the tumor cells, as this response functions as a critical innate antiviral mechanism and could explain the failure of FusOn-H2 to replicate well in some tumor lines but not others. For this purpose, we constructed a testing plasmid, pJ-ISRE-SEAP, in which SEAP gene is driven by a minimal promoter linked to 3 tandem repeats of ISRE, derived from the ISG56 promoter region. When transfected into tumor cells, this construct enabled us to measure SEAP levels in the culture medium and hence to monitor the cells' interferon response activity. We transfected this plasmid into the five lines of tumor cells that showed resistance to FusOn-H2 as well as a panel of tumor cells that are permissive to the virus. Supernatants were collected and the secreted SEAP quantified at different time points after transfection. All five resistant cell lines had much higher levels of SEAP secretion than did the permissive lines. In contrast, SEAP secretion was at very low level in cells fully permissive to FusOn-H2.

### Example 7

### Induction of Neutrophil Infiltration by FusOn-H2 is a General Phenomenon in Tumors Formed from Resistant Tumor Cells

In addition to the EC9706 tumor, we also investigated the ability of FusOn-H2 in inducing neutrophil infiltration in tumors formed from B16 melanoma cells that are also highly resistant to FusOn-H2 replication. The results show that a massive neutrophil infiltration was visible after treatment with FusOn-H2, but not with PBS. Thus, induction of neutrophil infiltration by FusOn-H2 is a general phenomenon in tumors from cells resistant to the virus replication.

What is claimed is:

1. A method of treating tumor cells in an HSV-based oncolytic virotherapy, comprising:
administering a therapeutic composition comprising a FusOn-H2 oncolytic virus.

2. The method of claim 1, wherein administration of the FusOn-H2 oncolytic virus increases an innate immune response to infected tumor cells.

3. The method of claim 1, wherein the FusOn-H2 oncolytic virus is administered to tumor cells resistant to lytic effects of oncolytic viruses.

4. The method of claim 1, wherein the FusOn-H2 oncolytic virus is administered to tumor cells permissive to lytic effects of oncolytic viruses.

5. The method of claim 2, wherein the innate immune response induces the infiltration of neutrophils in the tumor.

6. The method of claim 3, wherein the resistant tumor cells are at least one of esophageal carcinoma (EC9706), cervical cancer (Hela), lung carcinoma (LL2), pancreatic cancer (H7), or melanoma (B16).

7. The method of claim 4, wherein the permissive tumor cells are at least one of HuH-7, MCF-7, Miapaca2, PC-3, A549, 4T1, MD-MBA-435 or Hep G2.

8. The method of claim 3, wherein the resistant tumor cells comprise an endogenous interferon response.

9. The method of claim 1, further comprising screening a patient for tumor cells comprising an endogenous interferon response.

10. The method of claim 5, wherein the infiltration of neutrophils in the tumor kills the tumor cells.
